(19) United States
(12) Patent Application Publication
(10) Pub. No.: US 2019/0015553 A1
(43) Pub. Date: Jan. 17, 2019

(54) Preparation method of injectable extracellular matrix based hydrogel derived from decellularized porcine skin loaded with bi-phasic calcium phosphate

(71) Applicant: Soonchunhyang University Industry Academy Cooperation Foundation, Asan-si (KR)

(72) Inventors: Byong Taek Lee, Cheonan-si (KR); Reiza Ventura, Cheonan-si (KR); Andrew R. Padahin, Cheonan-si (KR)

(21) Appl. No.: 15/986,971

(22) Filed: May 23, 2018

(30) Foreign Application Priority Data
Jul. 12, 2017 (KR) 10-2017 -0088568

(51) Int. Cl. A61L 27/36; A61L 27/50; A61L 27/52; A61L 27/12; A61L 27/56

(52) U.S. Cl. CPC A61L 27/3633; A61L 27/50; A61L 27/52; A61L 2430/02; A61L 27/56; A61L 2400/06; A61L 27/12

(57) ABSTRACT

The present invention relates to a method for preparing an injectable extracellular matrix-based hydrogel. The injectable extracellular matrix-based hydrogel exhibits excellent biocompatibility, and superior cellular proliferation and bone regeneration via intercellular interaction, thus being effectively useful as a filler for bone regeneration. In addition, the injectable extracellular matrix-based hydrogel exhibits excellent porosity, has an interconnected structure and is thermogelling, based on thermosensitivity of showing a sol-gel transition depending on temperature, thus undergoing rapid gelation upon implantation in vivo and promoting bone regeneration.

### BACKGROUND OF THE INVENTION

### Field of the Invention

The present invention relates to a method for preparing an injectable extracellular matrix-based hydrogel including mixing an extracellular matrix-based hydrogel with a biphasic calcium phosphate powder to prepare an injectable extracellular matrix-based hydrogel containing biphasic calcium phosphate.

### Description of the Related Art

Bone tissues play an important role in maintaining the skeleton of the human body and various materials and forms of bone graft materials for replacing or reproducing bone tissues are being researched and developed. Bone graft materials can be classified into osteogenic materials, osteoconductive materials and bone-inductive materials according to the healing mechanism of bones. A method such as autograft, allograft or transplantation is commonly used depending on the implant materials used for bone transplantation or implantation.

Thereamong, autograft is a method capable of minimizing immune reactions and has advantages of minimizing immune reactions and thus regenerating stable bone tissues by implanting autogenous bones into damaged bone sites. However, autograft also has inconvenience such as secondary bone loss in other sites and long recovery period due to extraction of autogenous bones and a disadvantage of very limited amount of bones. In an attempt to compensate for this, transplantation using bones of other subjects may be used. However, disadvantageously, transplantation causes lots of immune reactions and is very expensive, unlike autograft. Therefore, a great deal of research is underway on bone tissue engineering to produce synthetic bones which can be applied to many patients while minimizing immune reaction and transplant synthetic bones into them.

Scaffolds for application to bone tissue engineering should meet some core requirements to form optimized tissues of host tissues. These conditions include cell affinity, appropriate porosity allowing for permeation of nutrients and oxygen, surface activity to promote cellular attachment and differentiation, and the like. In addition, ideally, scaffolds should be continuously decomposed and replaced by host cells.

For the treatment of bone defects due to injury, bone tumors and periodontal diseases, clinical need for bone scaffolds has been increasing worldwide. In addition, in an attempt to minimize problems such as limited availability of allogeneic bones, homogeneous bones and heterogeneous bones, donor site morbidity, immune rejection and infection, implantation of artificial bones is used. A variety of biomaterials such as artificial bones, ceramic polymers and mixtures thereof are being tested.

On the other hand, injectable hydrogels have advantages associated with transplant operation and can be replaced through a minimally invasive injection procedure. Injection-type tissue engineering has attracted much attention since it can minimize the risk of infection and scarring and reduce costs, and is used to fill irregularly sized defects which are commonly created by trauma, tumor resection or congenital defects.

Accordingly, the present invention has been completed, based on the finding that injectable extracellular matrix-based hydrogels, which are applicable to noninvasive methods, and are produced using decellularized porcine skin and a biphasic calcium phosphate powder, exhibit excellent biocompatibility, and superior cellular proliferation and bone regeneration via intercellular interaction.

### PRIOR ART

### Patent Document

(Patent Document) Korean Patent Laid-open No. 10-2011-0025530

### SUMMARY OF THE INVENTION

Therefore, the present invention has been made in view of the above problems, and it is one object of the present invention to provide a method for preparing an injectable extracellular matrix-based hydrogel including mixing an extracellular matrix-based hydrogel with a biphasic calcium phosphate powder to prepare an injectable extracellular matrix-based hydrogel containing biphasic calcium phosphate.

It is another object of the present invention to provide a filler for bone regeneration including the injectable extracellular matrix-based hydrogel prepared by the method.

The present inventors produced extracellular matrix (ECM)-based hydrogels by digesting lyophilized decellularized porcine skin with a pepsin-containing hydrochloric acid solution to obtain an extracellular matrix-containing homogeneous solution and adding sodium hydroxide to the extracellular matrix-containing homogeneous solution, produced injectable extracellular matrix-based hydrogels containing biphasic calcium phosphate by mixing the extracellular matrix-based hydrogels with biphasic calcium phosphate, identified that the injectable extracellular matrix-based hydrogels exhibit excellent biocompatibility, and superior cellular proliferation and bone regeneration via intercellular interaction, and identified that the injectable extracellular matrix-based hydrogels are gelled in vivo owing to thermosensitivity of showing a sol-gel transition depending on temperature and being used as a bone filler.

In accordance with the present invention, the above and other objects can be accomplished by the provision of a method for preparing an injectable extracellular matrix-based hydrogel including decellularizing and lyophilizing the porcine skin, digesting the lyophilized decellularized porcine skin with a hydrochloric acid solution containing pepsin to prepare an extracellular matrix-containing homogeneous solution, adding sodium hydroxide to the extracellular matrix-containing homogeneous solution to prepare an extracellular matrix-based hydrogel, and mixing the extracellular matrix-based hydrogel with a biphasic calcium phosphate powder to prepare an injectable extracellular matrix-based hydrogel containing biphasic calcium phosphate.

The method for preparing an injectable extracellular matrix-based hydrogel includes decellularizing and lyophilizing the porcine skin.

In the present invention, the porcine skin is decellularized using SDS dissolved in isopropanol and triton X-100 and then lyophilized.

The method for preparing an injectable extracellular matrix-based hydrogel according to the present invention may include digesting the lyophilized decellularized porcine skin with a pepsin-containing hydrochloric acid solution to prepare an extracellular matrix-containing homogeneous solution.

In an embodiment of the present invention, specifically, the lyophilized decellularized porcine skin is digested with a hydrochloric acid solution containing 1 mg of pepsin in 1 ml of 0.1M HCl for 48 hours until the extracellular matrix-containing homogeneous solution is obtained.

The method may include adding sodium hydroxide to the extracellular matrix-containing homogeneous solution of the injectable extracellular matrix-based hydrogel according to the present invention to prepare an extracellular matrix-based hydrogel.

In an embodiment of the present invention, specifically, 1M NaOH (sodium hydroxide) is added to the extracellular matrix (ECM)-containing homogeneous solution to adjust pH to 7 to 8, thereby preparing an extracellular matrix (ECM)-based hydrogel with a concentration of 30% (w/v) ECM.

The decellularized extracellular matrix (ECM) contains combined ECM ingredients for mimicking the ECM of original tissues and is highly biologically active for tissue remodeling and reproduction. In addition, decellularization can eliminate cell membrane antigens and nuclear components to minimize immune reactions and thereby make materials safe for use in clinical practice.

The method for preparing an injectable extracellular matrix-based hydrogel according to the present invention may include mixing the extracellular matrix-based hydrogel with a biphasic calcium phosphate powder to produce an injectable extracellular matrix-based hydrogel containing biphasic calcium phosphate.

Biphasic calcium phosphate (BCP) is a mixture of two different calcium phosphate phases: the sparingly soluble hydroxyapatite (HA) and highly soluble tricalcium phosphate in different ratios and degradation kinetics thereof can be tuned both in vitro and in vivo.

According to the present invention, the extracellular matrix-based hydrogel comprises the biphasic calcium phosphate powder in an amount of 12 to 18% (w/v), more specifically, 15% (w/v).

In an embodiment of the present invention, the extracellular matrix-based hydrogel is mixed with a biphasic calcium phosphate (BCP) powder in an amount of 15% (w/v) BCP to prepare an injectable extracellular matrix-based hydrogel containing biphasic calcium phosphate.

The injectable extracellular matrix-based hydrogel according to the present invention can be gelled in vivo at a body temperature due to thermosensitivity of showing a sol-gel transition depending on temperature, when applied as a bio-material in vivo.

In addition, since the injectable extracellular matrix-based hydrogel is a biodegradable ingredient and has thermosensitivity of exhibiting sol-gel behavior depending on temperature, it can be easily injected as a solution into the body to form a three-dimensional gel within a short time due to body temperature.

In addition, the injectable extracellular matrix-based hydrogel can be utilized in various applications requiring strength such as tissues engineering materials, for example, implant materials and artificial cartilage, and can be practically applicable to the human body.

The injectable extracellular matrix-based hydrogel prepared by the method is in a sol at 25° C. or less and in a gel at 37° C.

In an embodiment of the present invention, when the injectable ECM hydrogel is transplanted in vivo, gelation effectively occurs in rabbit's femoral head, which indicates that gelation effectively occurs in vivo at 37° C., the body temperature.

In addition, the injectable extracellular matrix-based hydrogel prepared according to the present invention has a porous structure.

In addition, as the content of biphasic calcium phosphate powder added to the injectable extracellular matrix-based hydrogel increases, gelation of the injectable extracellular matrix-based hydrogel becomes faster.

In an embodiment of the present invention, it can be confirmed that the injectable extracellular matrix-based hydrogel has a porous structure and exhibits excellent interconnectivity with cells, and as the content of the biphasic calcium phosphate increases, gelation of the injectable extracellular matrix-based hydrogel becomes faster.

Specifically, in the case of ECM-15% BCP, gelation effectively occurs in vivo, but in the case of implantation of ECM-10% BCP, gelation does not effectively occur in vivo, indicating the ECM-10% BCP hydrogel is unsuitable as an injectable extracellular matrix-based hydrogel.

In addition, results of bone formation in defect sites after implantation of injectable extracellular matrix-based hydrogels revealed that ECM-0% BCP (50.51±15.44) and ECM-15% BCP (68.04±15.95) as injectable extracellular matrix-based hydrogels have excellent bone formation, as compared to the negative control (33.28±13.05), and ECM-15% BCP containing biphasic calcium phosphate has excellent bone formation, as compared to ECM-0% BCP containing no biphasic calcium phosphate.

In addition, in an embodiment of the present invention, the injectable extracellular matrix-based hydrogel is well integrated with the host bone with new bone formation starting from the periphery of the defect all the way to the center.

Accordingly, the injectable extracellular matrix-based hydrogel exhibits excellent biocompatibility, and superior cellular proliferation and bone regeneration via intercellular interaction, thus being effectively useful as a filler for bone regeneration. In addition, the injectable extracellular matrix-based hydrogel exhibits excellent porosity, has an interconnected structure and undergoes thermogelling, thus inducing rapid gelation upon implantation in vivo and promoting bone regeneration.

In another aspect of the present invention, provided is a filler for bone regeneration including the injectable extracellular matrix-based hydrogel prepared by the method.

As described above, the injectable extracellular matrix-based hydrogel effectively promotes bone regeneration and is thus applicable as a bone regeneration filler.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows injectable hydrogels of ECM-0% BCP, ECM-10% BCP and ECM-15% BCP in liquid form at 4° C. and in gel form at 37° C.;

FIG. 2 shows low and high magnification scanning electron micrographs of ECM-0% BCP (A.1-A.2), ECM-10% BCP (B.1-B.2) and ECM-15% BCP (C.1-C.2) and an EDS profile of a representative sample of injectable ECM with a BCP powder (D);

FIG. 3 shows % total porosity of injectable ECM hydrogels (A) and corresponding pore sizes of ECM-0% BCP (B), ECM-10% BCP (C) and ECM-15% BCP (D);

FIG. 4 shows turbidimetric gelation (A) of ECM-0% BCP, ECM-10% BCP and ECM-15% BCP determined spectrophotometrically by measuring absorbance during gelation. Kinetic parameters such as tlag, lag time of gelation (B), t½, half gelation time (C) and s, and speed of gelation (D) were calculated based on turbidimetric gelation values;

FIG. 5 shows cell viability assay (A) of RBMSC exposed to 1-day and 7-day extracts after 24 hour exposure, and rBMSC distribution (B) cultured on ECM-0% BCP, ECM-10% BCP and ECM-15% BCP after 7 days;

FIG. 6 shows gelation of ECM-0% BCP and ECM-15% BCP upon implantation in rabbit's femoral head;

FIG. 7 are micro-CT micrographs showing new bone formation in femoral head defects after implantation of ECM-0% BCP and ECM-15% BCP compared with negative control; and

FIG. 8 shows H&E stained tissue sections with bone formation on negative control, ECM-0% BCP and ECM-15% BCP implanted in rabbit's femoral head after 4 weeks.

### DETAILED DESCRIPTION OF THE INVENTION

Hereinafter, examples of the present invention will be described with reference to the annexed drawings in detail to such an extent that a person having ordinary knowledge in the art to which the present invention pertains can easily implement the examples. However, the present invention can be realized in various forms and is not limited to the examples described herein.

### Example 1: Production of Injectable Extracellular Matrix-Based Hydrogel

In the present invention, an injectable hydrogel for bone regeneration derived from decellularized porcine skin was produced.

First, the porcine skin was purchased and then washed with deionized water, the epidermis was thinly cut with a scalpel blade, was ground with a mixer and washed with water to remove coagulated fats, the residue was centrifuged at 3,500 rpm for 5 minutes, the upper layer containing fats was discarded and only the precipitated epidermis layer was used. The epidermis layer was reacted with 0.25% trypsin solution at 37° C. for 6 hours and washed with water again. Then, the resulting product was reacted in 70% isopropanol solution containing 0.1% SDS at 37° C. for 6 hours and then reacted in a 70% isopropanol solution containing 1% triton X-100 at 37° C. for 12 hours. The 70% isopropanol solution containing 1% triton X-100 was changed every 4 hours. Then, the resulting product was washed five times with fresh water and then reacted in 100% isopropanol for 12 hours at 37° C. while stirring. The 100% isopropanol solution was changed every 4 hours. Then, the resulting product was washed with water until volatile odor was eliminated and lyophilized for 3 days which was then used.

Then, a lyophilized decellularized extracellular matrix (ECM) powder was digested for 48 hours in an HCl solution containing 1 mg of pepsin (Sigma) per 1 ml of 0.1M HCl until a homogeneous solution was obtained to prepare a homogeneous solution containing an extracellular matrix. Sodium hydroxide was added to the extracellular matrix-containing homogeneous solution to adjust pH to 7.4, leaving a final concentration of 30% (w/v) ECM.

In addition, the extracellular matrix-based hydrogel was mixed with a biphasic calcium phosphate powder to prepare an injectable extracellular matrix-based hydrogel containing biphasic calcium phosphate. Specifically, as control, ECM-0% BCP not mixed with a biphasic calcium phosphate powder was prepared, and ECM-10% BCP wherein 0.10 g of a BCP powder (10% w/v BCP powder) is added to 1 mL of the extracellular matrix-based hydrogel, and ECM-15% BCP wherein 0.15 g of a BCP powder (15% w/v BCP powder) is added to 1 mL of the extracellular matrix-based hydrogel were prepared.

### Example 2: Rat Bone Marrow Mesenchymal Stem Cell Culture

Rat bone marrow mesenchymal stem cells (rBMSC) were isolated according to a well-known method. Aspirates of BMSC were isolated from rat femurs (Samtako Bio, Korea), expanded in alpha-minimum essential medium (αMEM, Life Technologies) supplemented with 10% fetal bovine serum (FBS, Life Technologies) and 1% penicillin-streptomycin (PS, Life Technologies) and cultured at 37° C. in a 5% CO₂ atmosphere. Third passage cells were used in subsequent experiments.

### Example 3: Cytotoxicity Analysis

Cytotoxicity of the injectable extracellular matrix-based hydrogel prepared in Example 1 was assessed by cell viability test. rBMSCs were plated and grown to 80% confluency prior to cell viability assay. Liquid extracts were prepared by incubating 1 ml of the injectable extracellular matrix-based hydrogel in appropriate media for to 7 days. Cells were trypsinized and plated at a concentration of 2×10⁴ cells/well in a 24-well plate. After hours, a culture medium was replaced with liquid extracts. Then, the cells were incubated at 37° C. under 5% CO₂ for 24 hours prior to evaluation of viable cells via MTT metabolic assay.

100 μl of a MTT reagent, 3-(4,5-dimethylthiazol-2-yl)-2,5-diphenyltetrazolium bromide (Sigma Aldridge, USA), was added to each well and incubated for 4 hours to develop purple formazan salts. Then, media and MTT solution were removed and DMSO (Samchun Pure Chemical Co., LTD., Korea) was added to each well, followed by incubation at room temperature for 1 hour to dissolve formazan salts. The resulting purple formazan salts were transferred to 96-well plates. The absorbance of each well was measured at a wavelength of 595 nm using a microplate reader. Metabolically active and viable cells reduced MTT into purple colored formazan salts while dead cells did not have the ability to convert MTT into formazan. Optical density with an absorbance of 500 to 600 nm corresponded to the number of viable cells.

### Example 4: Fluorescence Imaging

Distribution and morphology of rBMSC seeded onto the injectable extracellular matrix-based hydrogels were evaluated using F-actin staining. Injectable extracellular matrix-based hydrogels were prepared by injecting 500 μl samples into a 24-well plate using a 25 gauge needle. An rBMSC suspension with a concentration of 2×10⁴ cell/mL was seeded onto the surface of the injectable extracellular matrix-based hydrogels and allowed to grow for 1 to 7 days. A culture medium was changed every 2 days. Samples were then fixed with 4% paraformaldehyde after a predetermined time, permeabilized with 10% Triton-X (sigma Aldrich, USA), washed with phosphate buffered saline (PBS; Sigma Aldrich, USA) to remove residual Triton-X, blocked with 1% bovine serum albumin (BSA; Sigma Aldrich, USA) and stained with fluorescein isothiocyanate-conjugated phalloidin (FITC, Sigma-Aldrich) to visualize the actin filaments, and nuclei were counterstained with Hoechst 33342 (Sigma-Aldrich). Micrographs were taken using Fluoview FV10i (Olympus, Japan) and analyzed with FV10i-ASW 3.0 viewer software to determine cell distribution.

### Example 5: Animals and Surgical Procedure

All procedures were performed according to the guidelines of the Animal Care Center of Soonchunhyang University, Chungcheongnam-do, South Korea. New Zealand white rabbits (Samtaco, n=12) were divided into three groups during a period of 4 weeks; negative control with defects only, and positive controls of ECM-0% BCP and ECM-15% BCP. Rabbits were anaesthetized using isoflurane (Piramal, USA). The right foreleg was shaved, cleaned with 75% ethanol and sterilized with an iodine solution. The femoral head was vertically incised. The skin was retracted and the subcutaneous skin was incised to expose the femoral head. A defect with a size of 6 mm×5 mm was made using a trephine drill while occasionally washing with saline to prevent tissue dehydration and heating. ECM-0% BCP (n=4) or ECM-15% BCP (n=4) was injected into the defect site, while the negative control (n=4) was not treated. The incisions were closed with sutures disinfected by an iodine solution. Then, the animal subjects were returned to their cages and allowed to recover with ad libitum food and water. Scaffolds were retrieved after 4 weeks by sacrificing the animals and extracting the implanted samples.

### Example 6: Evaluation of Micro-Computed Tomography (Micro-CT)

Extracted femur heads were cut, fixed overnight with paraformaldehyde, washed with running water for 10 min and covered with paraffin. The femur heads were scanned with a 1076 mCT scanner (Skyscan). Bone volume/tissue volume (BV/TV) was calculated with C Tan (Skyscan) and 3D reconstructions were created using Seg3D (Scientific Computing and Imaging Institute) software.

### Example 7: Histological Observation

Tissue sections of extracted samples were prepared to examine the extent of bone formation respectively. Samples were decalcified using a 5% nitric acid solution dehydrated in alcohols, soaked in xylene, and embedded in paraffin for sectioning. Then, samples were cut into 5-mm sections, stained with H&E (Sigma Aldrich) according to manufacturer's protocol and visualized under an Olympus bright field microscope using a camera.

### Test Example 1: Analysis of Injectable Extracellular Matrix-Based Hydrogel

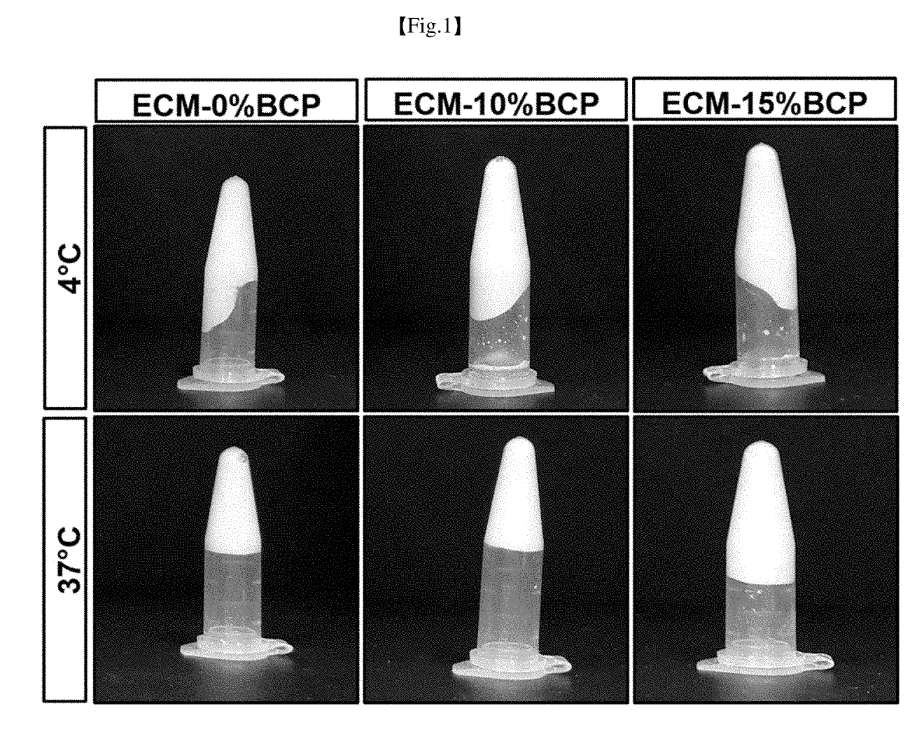

In Example 1, an extracellular matrix-containing homogeneous solution derived from porcine skin was fabricated using pepsin digestion and pH of the resulting solution was adjusted to pH 7.4 with 1M NaOH. FIG. 1 shows ECM-0% BCP, ECM-10% BCP and ECM-15% BCP hydrogels present in a liquid form at 4° C. and in a gel at 37° C., and represents thermogelling properties of the samples. That is, this demonstrates that the samples have thermosensitivity of showing a sol-gel transition depending on temperature.

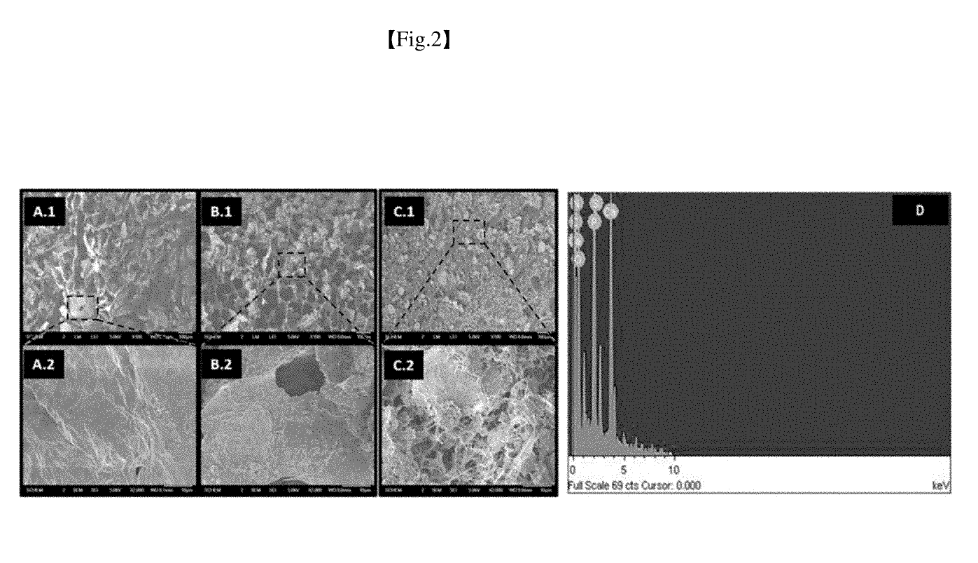

FIG. 2 shows low-magnification and high-magnification SEM images of ECM-0% BCP (A.1-A.2), ECM-10% BCP (B.1-B.2) and ECM-15% BCP (C.1-C.2) and an EDS profile (D) of representative samples of injectable extracellular matrix-based hydrogels to which a BCP powder is added.

Low-magnification SEM images of ECM-0% BCP (A.1), ECM-10% BCP (B.1) and ECM-15% BCP (C.1) in FIG. 2 showed porous constructs with high interconnectivity. High-magnification SEM images showed the rough surface of ECM-0% BCP (A.2), BCP powder embedded into ECM-10% BCP making the surface rougher (B.2) and formation of micropores with collagen fibers was more evident on the ECM-15% BCP (C.2).

The EDS profile showed that elemental peaks of carbon, nitrogen, phosphorus and sulfur which are usually found on decellularized ECM and peaks of BCP powder which consist of carbon, oxygen and phosphorus. These results indicated that the BCP powder was successfully incorporated into injectable ECM hydrogels.

### Test Example 2: Analysis of Porosity and Pore Size of Injectable Extracellular Matrix-Based Hydrogels

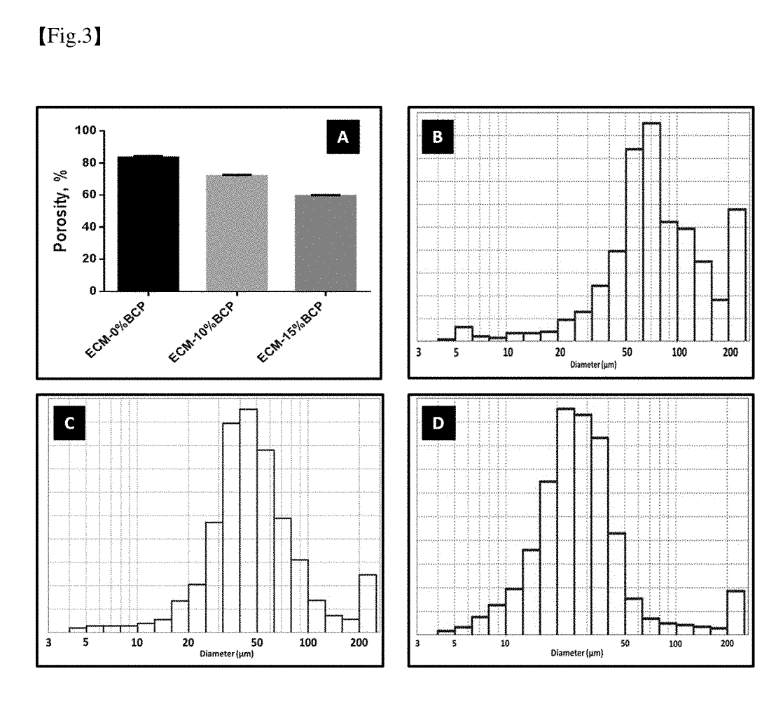

FIG. 3 shows that total porosity (A) and the corresponding pore size of ECM-0% BCP (B), ECM-10% BCP (C) and ECM-15% BCP (D) injectable extracellular matrix-based hydrogels, with an increase in BCP powder. As a result, as BCP content increases, porosity decreases and pore size decreases.

### Test Example 3: Analysis of Tubidimetric Gelation Kinetics of Injectable Extracellular Matrix-Based Hydrogels

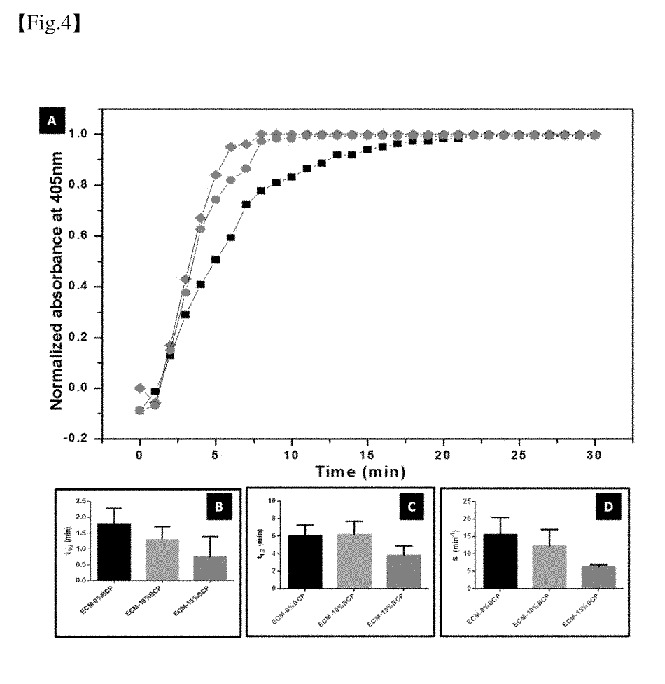

FIG. 4 shows normalized tubidimetric gelation kinetics of ECM-0% BCP, ECM-10% BCP and ECM-15% BCP, and gelation kinetics were calculated. These results indicate that gelation occurred after lag phase. Gelation kinetics such as tlag, t½ and S decreased as the content of a BCP powder increased. The results indicate that the addition of BCP powder induced faster gelation of injectable extracellular matrix-based hydrogels.

Specifically, when applied from 4° C. to 37° C., it took about 15 minutes for ECM-0% BCP to change from sol to gel, it took about 12 minutes for ECM-10% BCP to change from sol to gel, and it took about 7 minutes for ECM-15% BCP to change from sol to gel (FIG. 4D). In conclusion, as the content of BCP powder increase, gelation is facilitated.

### Test Example 4: Cytotoxicity Analysis

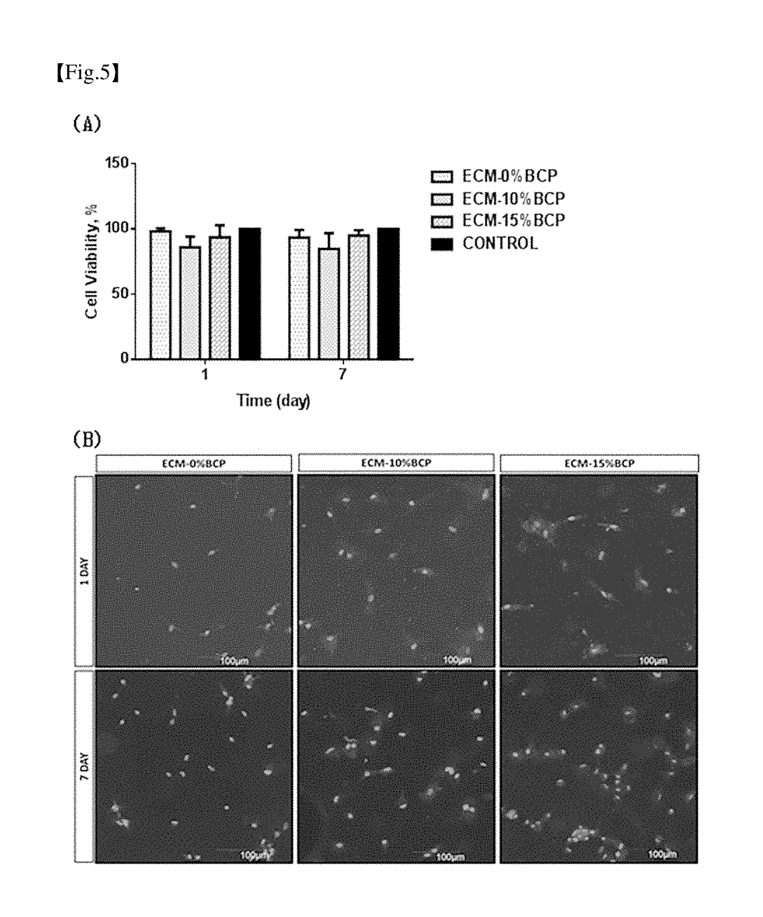

rBMSC viability and distribution were assessed using MTT and fluorescent imaging, as shown in FIG. 5. Viability of rBMSC after exposure to 1 and 7 day extracts for 24 hours revealed that injectable extracellular matrix-based hydrogels neither exhibited toxicity nor contained toxic byproducts (FIG. 5A). Cell distributions of Hoechst 33342 and FITC stained samples showed increased rBMSC with widely spread filaments in all of injectable ECM-based hydrogels after 7 days of culture.

### Test Example 5: Evaluation of Bone Regeneration

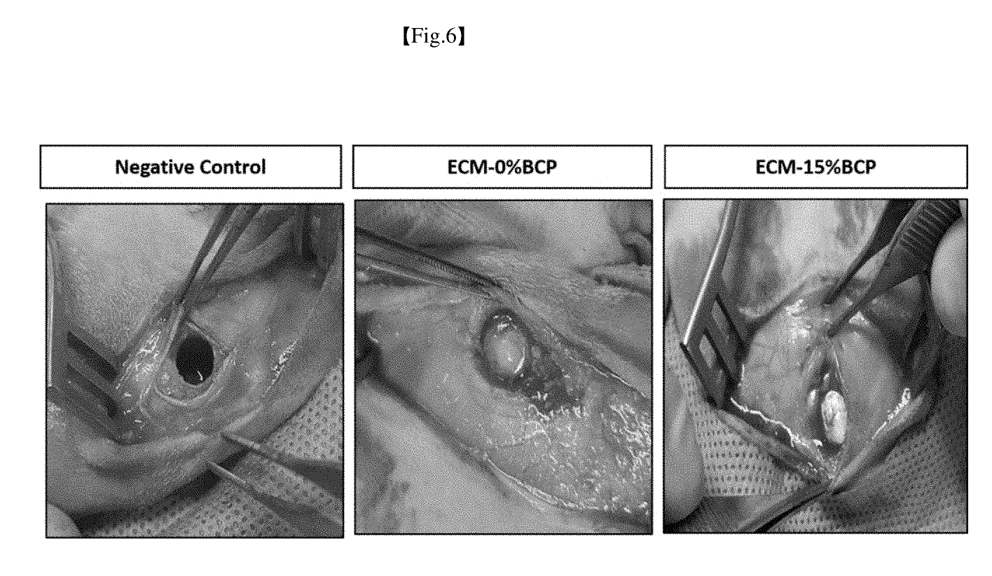

FIG. 6 shows gelation of ECM-0% BCP and ECM-15% BCP observed upon implantation in the rabbit's femoral head, indicating that the injectable extracellular matrix-based hydrogels were effectively gelled in vivo, unlike the negative control. On the other hand, upon implantation of ECM-10% BCP prepared in Example 1, the injectable extracellular matrix-based hydrogels were not effectively gelled in vivo, unlike ECM-15% BCP, making it unsuitable as injectable extracellular matrix-based hydrogels.

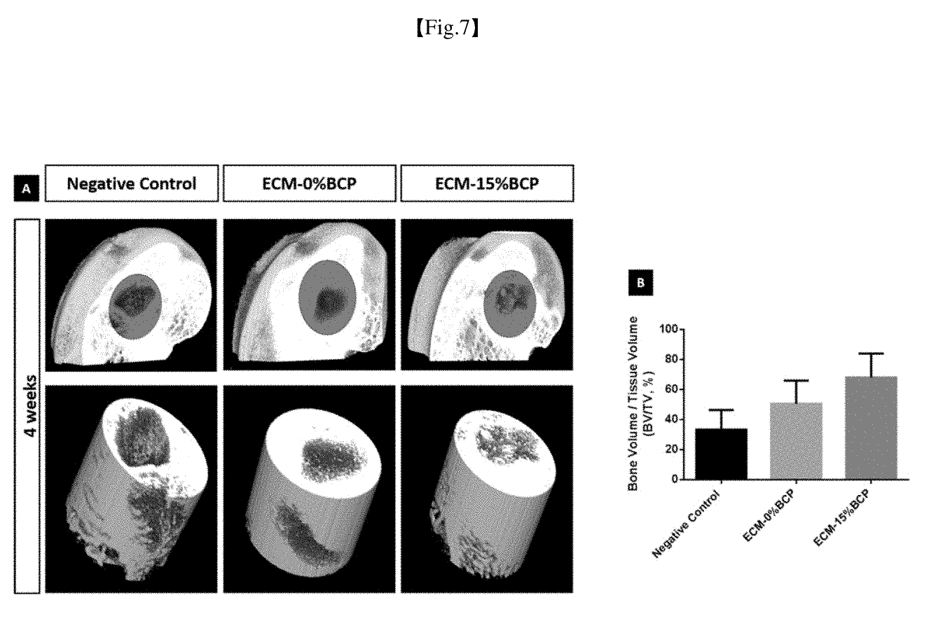

FIG. 7 shows micro-CT micrographs of negative control, ECM-0% BCP and ECM-15% BCP with new bone formation at the defect site 4 weeks after implantation. Quantification assay of bone volume/tissue volume revealed that higher bone formation was observed in ECM-0% BCP (50.51±15.44) and ECM-15% BCP (68.04±15.95) compared with negative control (33.28±13.05). Bone formation was the most effective in ECM-15% BCP containing BCP.

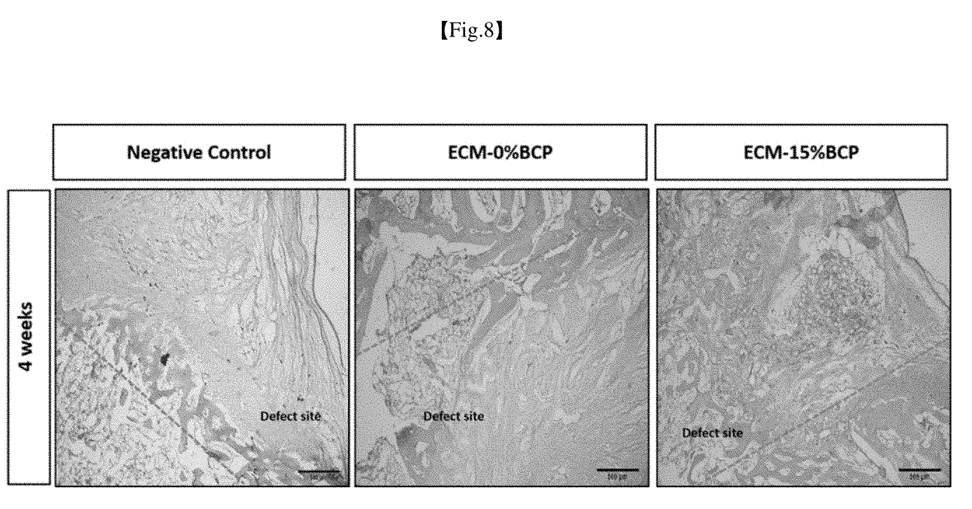

FIG. 8 shows H&E stained tissue sections with bone formation of negative control, ECM-0% BCP and ECM-15% BCP implanted in rabbit's femoral head after 4 weeks. As a result, injectable extracellular matrix-based hydrogels were well integrated with the host bone with new bone formation starting from the periphery of the defect all the way to the center (FIG. 8). No adverse reaction was also observed in all of the samples upon in vivo implantation.

The present invention relates to a method for preparing an injectable extracellular matrix-based hydrogel. The injectable extracellular matrix-based hydrogel exhibits excellent biocompatibility, and superior cellular proliferation and bone regeneration via intercellular interaction, thus being effectively useful as a filler for bone regeneration. In addition, the injectable extracellular matrix-based hydrogel exhibits excellent porosity, has an interconnected structure and is thermogelling, thus inducing rapid gelation upon implantation in vivo and promoting bone regeneration.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for preparing an injectable extracellular matrix-based hydrogel comprising:
decellularizing and lyophilizing porcine skin;
digesting the lyophilized decellularized porcine skin with a hydrochloric acid solution containing pepsin to prepare an extracellular matrix-containing homogeneous solution;
adding sodium hydroxide to the extracellular matrix-containing homogeneous solution to prepare an extracellular matrix-based hydrogel; and
mixing the extracellular matrix-based hydrogel with a biphasic calcium phosphate powder to prepare an injectable extracellular matrix-based hydrogel containing biphasic calcium phosphate.

2. The method according to claim 1, wherein the injectable extracellular matrix-based hydrogel has thermosensitivity of showing a sol-gel transition depending on temperature.

3. The method according to claim 1, wherein the injectable extracellular matrix-based hydrogel is gelled in vivo.

4. The method according to claim 1, wherein the extracellular matrix-based hydrogel comprises the biphasic calcium phosphate powder in an amount of 12 to 18% (w/v).

5. The method according to claim 1, wherein the extracellular matrix-based hydrogel comprises the biphasic calcium phosphate powder in an amount of 15% (w/v).

6. The method according to claim 1, wherein the injectable extracellular matrix-based hydrogel has a porous structure.

7. The method according to claim 1, wherein the injectable extracellular matrix-based hydrogel facilitates bone regeneration.

8. A filler for bone regeneration comprising the injectable extracellular matrix-based hydrogel prepared by the method according to claim 1.
